(19) United States
(12) Patent Application Publication
(10) Pub. No.: US 2022/0380678 A1
(43) Pub. Date: Dec. 1, 2022

(54) TERPENE ESTER SURFACTANTS

(71) Applicant: P2 Science, Inc., Woodbridge, CT (US)

(72) Inventor: Patrick FOLEY, New Haven, CT (US)

(21) Appl. No.: 17/746,624

(22) Filed: May 17, 2022

Related U.S. Application Data
(60) Provisional application No. 63/189,543, filed on May 17, 2021.

(51) Int. Cl. C09K 23/36; C09K 23/34; C07C 69/675; C07C 69/42; C07C 69/34; C07C 67/08

(52) U.S. Cl. CPC C09K 23/36; C09K 23/34; C07C 69/675; C07C 69/42; C07C 69/34; C07C 67/08

(57) ABSTRACT

The present disclosure is directed to novel derivatives of terpenes, particularly ester derivatives of terpene alcohols, and methods of making them, compositions comprising them, and methods for using them.

### CROSS-REFERENCE TO RELATED APPLICATIONS

This U.S. non-provisional application claims priority to, and the benefit of, U.S. Provisional Application No. 63/189,543, filed on May 17, 2021, the contents of which are hereby incorporated by reference in its entirety.

### FIELD OF INVENTION

The present disclosure is directed to novel derivatives of terpenes, particularly ester derivatives of terpene alcohols, and methods of making them, compositions comprising them, and methods for using them.

### BACKGROUND

Terpenes and terpene derivatives constitute one of the most diverse, commercially sought after, and industrially important classes of natural products. Terpenes occur in all organisms and are particularly prevalent in plants, from which they are industrially isolated. The ready commercial access and low-cost of terpenes continually drives innovation into their chemical derivatization which find use in polymer science, the flavor & fragrance industry, the cosmetic industry, the pharmaceutical industry, and as surfactants, plastic additives, and other industrial uses.

While base terpenes are inexpensive and widely available (C₅ₙH₈ₙ derivatives, n=1, 2, 3, etc.), chemically functionalized terpenes (terpenoids) are more useful, especially terpene alcohols. Common monoterpene alcohols include the following:

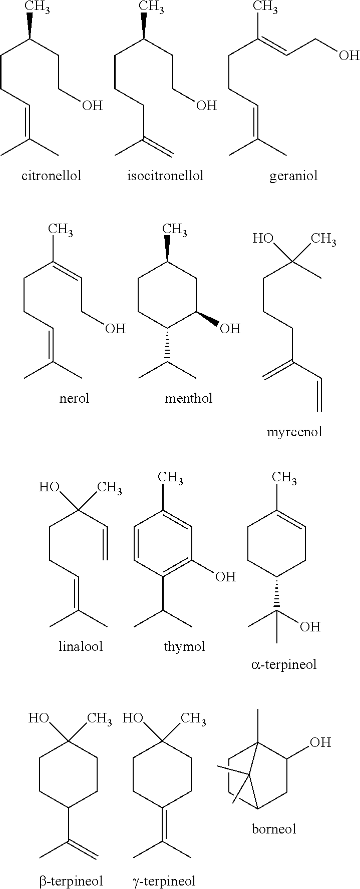

In addition to monoterpene alcohols, there are also inexpensive and widely available sesquiterpene alcohols, such as:

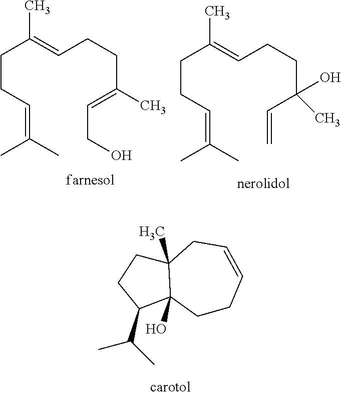

Terpene alcohol derivatives also include polymers and oligomers of terpene alcohols. For example, citronellol has been formed into useful oligomeric and polymeric products having the following structure:

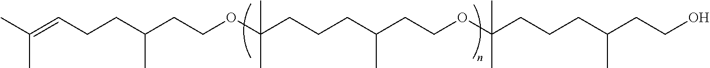

wherein n: 0-20 (e.g., 0-3). Dimers, trimers, and other oligomers of citronellol have been described. See, e.g., US2017/0283553, US2020/0165383, and US2020/0392287, the contents of each of which are hereby incorporated by reference in their entireties.

Surfactants are a multimillion-dollar annual industry. Surfactants are generally molecules which have at least one strongly polar or ionic functional group and at least one strongly nonpolar or hydrophobic functional group, generally positioned at opposite ends of a largely linear molecule. Thus, one end of a surfactant molecule is usually hydrophilic, while the other end is hydrophobic. The unique chemical property of surfactants is their ability to dissolve in both aqueous media and nonpolar (hydrophobic) media by virtue of this amphiphilic divided structure. They find use in a variety of products, often at the junction between aqueous and hydrophobic environments, especially when they are used in soaps and detergents. Functionally, surfactants are often used as wetting agents, detergents, soaps, emulsifiers, foaming agents, and dispersants. They are also commonly used in a variety of multicomponent compositions in order to stabilize the interactions between hydrophobic and hydrophilic ingredients.

There are a variety of different classes of commercially available surfactants, most of which are derived from petrochemical feedstocks. These include anionic surfactants such as sulfates, sulfonates, phosphates, and carboxylates; cationic surfactants, such as quaternary amines; zwitterionic surfactants, such as betaines, sultaines, and amine oxides; and non-ionic surfactants, such as ethoxylates, and polyhydroxy fatty acid esters.

There is an urgent need, however, for new surfactants, especially surfactants with improved stability, improved biodegradability, or improved environmental impact. It would be especially advantageous to have new surfactants sourced from renewable resources.

### BRIEF SUMMARY OF THE INVENTION

The present disclosure provides terpene alcohol ester surfactants, derived from terpene alcohols, and oligomers and derivatives thereof, and polar carboxylic acids, such as citric acid, ascorbic acid, glutaric acid, ascorbic acid, and gluconic acid, and derivatives thereof. These compounds are useful in numerous types of compositions, and numerous roles. For example, these compounds may be used as wetting agents, detergents, soaps, emulsifiers, foaming agents, dispersants, buffers, preservatives, and are especially useful as ingredients in personal care compositions, cosmetic compositions, and food compositions.

In a second aspect, the present disclosure provides a method of preparing such compounds.

In a third aspect, the present disclosure provides compositions and products comprising such compounds. In some embodiments, said compounds are useful in a variety of applications, including as or in cosmetics, soaps, hair care products, fragrances, sunscreens, plastic additives, paints, coatings, lubricants, and surfactants.

### DETAILED DESCRIPTION OF THE INVENTION

As used herein, the term “terpene alcohol” refers to a naturally terpene or terpenoid having or modified to have at least one alcohol functionality. The term includes both naturally occurring terpene alcohols, and alcohols derived from naturally occurring terpenes, such as by double bond oxidation, ketone reduction, or the like. As used herein, the term “terpene derivative” or “terpene alcohol derivatives” includes saturated and partially saturated derivatives of terpenes and terpene alcohols. Terpenes, terpene alcohols and other terpenoids commonly have 1, 2, 3 or more double bonds. In a saturated derivative all double bonds are hydrogenated, while in a partially saturated derivative, at least one double bond is hydrogenated, but at least one double bond is not. In this context, the double bonds of an aromatic ring are included; thus, a benzene ring can be considered to be partially saturated to form a cyclohexadiene or a cyclohexene ring, or fully saturated to form a cyclohexane ring.

In a first aspect, the present disclosure provides terpene alcohol ester surfactant compound (Compound 1) of the general formula (I):

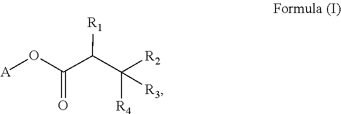

in free or salt form, wherein A is the core of a terpene alcohol or derivative thereof, and wherein R₁ is selected from hydrogen, carboxy (—COOH), carboxylato (—COO⁻), and hydroxy (—OH), or wherein R₁ and the methine carbon to which it is attached (CH—R₁) form a carbonyl (—C═O); and wherein R₂, R₃, and R₄ are each independently selected from:
H, C₁-C₁₂ alkyl, C₂-C₁₂ alkenyl, C₂-C₁₂ alkynyl, C₁-C₁₂ alkoxy, C₂-C₁₂ alkenyloxy, C₂-C₁₂ alkynyloxy, C₅-C₂₀ aryloxy, acyl (including C₂-C₁₂ alkylcarbonyl (—CO-alkyl) and C₆-C₂₀ arylcarbonyl (—CO-aryl)), acyloxy (—O-acyl), C₂-C₁₂ alkoxycarbonyl (—(CO)—O-alkyl), C₆-C₂₀ aryloxycarbonyl (—(CO)—O-aryl), C₂-C₁₂ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-N-substituted C₁-C₁₂ alkylcarbamoyl (—(CO)—NH(C₁-C₁₂ alkyl)), di-N-substituted alkylcarbamoyl (—(CO)—N(C₁-C₁₂ alkyl)₂), mono-N-substituted arylcarbamoyl (—(CO)—NH-aryl), halo (—F, —Cl, —Br, or —I), hydroxy (—OH), cyano (—C≡N), amino (—NH₂), mono- and di-N—(C₁-C₁₂ alkyl)-substituted amino, mono- and di-N—(C₅-C₂₀ aryl)-substituted amino, C₂-C₁₂ alkylamido (—NH—(CO)-alkyl), C₅-C₂₀ arylamido (—NH—(CO)-aryl), imino (—CR^{a}═NH where R^{a} is selected from hydrogen, C₁-C₁₂ alkyl, C₅-C₂₀ aryl, C₆-C₂₀ alkaryl, C₆-C₂₀ aralkyl, etc.), alkylimino (—CR^{b}═N(alkyl), wherein R^{b} is selected from hydrogen, alkyl, aryl, alkaryl, etc.), arylimino (—CR^{c}═N(aryl), where R^{c} is selected from hydrogen, alkyl, aryl, alkaryl, etc.), nitro (—NO₂), C₁-C₁₂ alkylsulfonyl (—SO₂-alkyl), and C₅-C₂₀ arylsulfonyl (—SO₂-aryl);
   wherein each of the aforementioned hydrocarbyl moieties of the preceding substituents, such as C₁-C₁₂ alkyl, C₂-C₁₂ alkenyl, C₂-C₁₂ alkynyl, and C₅-C₂₀ aryl, are each independently optionally further substituted by one or more groups selected from C₁-C₁₂ alkyl, C₂-C₁₂ alkenyl, C₂-C₁₂ alkynyl, C₁-C₁₂ alkoxy, C₂-C₁₂ alkenyloxy, C₂-C₁₂ alkynyloxy, C₅-C₂₀ aryloxy, acyl (including C₂-C₁₂ alkylcarbonyl (—CO-alkyl) and C₆-C₂₀ arylcarbonyl (—CO-aryl)), acyloxy (—O-acyl), C₂-C₁₂ alkoxycarbonyl (—(CO)—O-alkyl), C₆-C₂₀ aryloxycarbonyl (—(CO)—O-aryl), C₂-C₁₂ alkylcarbonato (—O—(CO)—O-alkyl), C₆-C₂₀ arylcarbonato (—O—(CO)—O-aryl), carboxy (—COOH), carboxylato (—COO⁻), halo (—F, —Cl, —Br, or —I), hydroxy (—OH), cyano (—C≡N), and amino (—NH₂);

provided that, at least one of R₁, R₂, R₃, or R₄ is a polar or ionic group (e.g., at least one of R₁, R₂, or R₃ is —OH, —COO⁻, or —COOH, or at least R₄ is or comprises a group selected from —OH, —COOH, —NH₂, or —NO₂.

In a preferred embodiment, the compound of Formula I is an isodecyl ester (i.e., group A is an isodecyl group).

It is understood that in the phrase “A is the core of a terpene alcohol or derivative thereof, that the terpene alcohol, or derivative thereof, from which the compound of Formula I is derived has the formula A-OH. This, the ester functional group of the compound of Formula I is formed, or is formable by, the condensation reaction as follows:

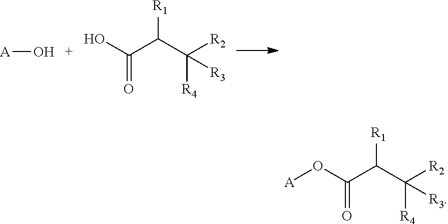

In further embodiments of the first aspect, the present disclosure provides as follows:
1.1 Compound 1, wherein A is the core of a terpene alcohol, or derivative thereof, wherein said terpene is a monoterpene, sesquiterpene, diterpene, sesterterpene, or triterpene.
1.2 Compound 1, wherein A is the core of a terpene alcohol, or derivative thereof, wherein said terpene is a monoterpene or sesquiterpene.
1.3 Compound 1, wherein A is the core of a terpene alcohol, or derivative thereof, wherein said terpene is a monoterpene (e.g., A is an isodecyl moiety).
1.4 Compound 1, wherein A is the core of a terpene alcohol, or derivative thereof, wherein said terpene alcohol is selected from citronellol, isocitronellol, geraniol, nerol, menthol, myrcenol, linalool, thymol, α-terpineol, β-terpineol, γ-terpineol, borneol, farnesol, nerolidol, and carotol.
1.5 Compound 1.4, wherein said terpene alcohol is selected from citronellol, geraniol, nerol, myrcenol, linalool, and farnesol.
1.6 Compound 1.5, wherein said terpene alcohol is selected from citronellol, myrcenol, linalool, and farnesol.
1.7 Compound 1, wherein A is the core of a terpene alcohol, or derivative thereof, wherein said terpene alcohol, or derivative, is an oligomer of citronellol.
1.8 Compound 1 or any of 1.1-1.7, wherein said terpene alcohol, or derivative thereof, has its natural unsaturation.
1.9 Compound 1 or any of 1.1-1.7, wherein said terpene alcohol, or derivative thereof, is partially unsaturated (e.g., monounsaturated or diunsaturated).
1.10 Compound 1 or any of 1.1-1.7, wherein said terpene alcohol, or derivative thereof, is fully saturated (e.g., said terpene alcohol is a fully saturated monoterpene derivative, e.g., an isodecyl moiety).
1.11 Compound 1, wherein A is selected from the group consisting of:

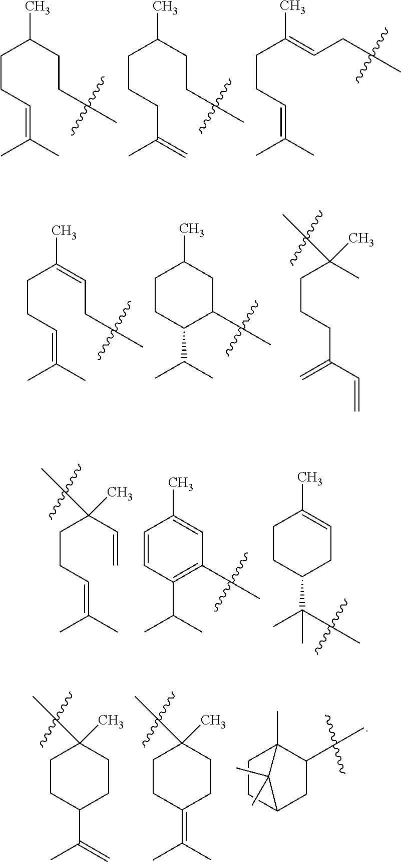

1.12 Compound 1, wherein A is selected from the group consisting of:

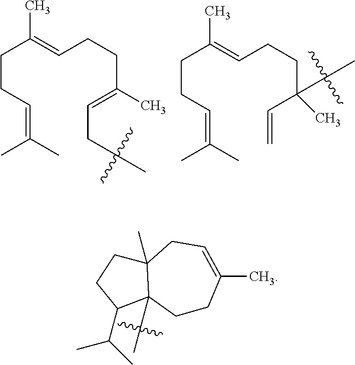

1.13 Compound 1, wherein A is selected from the group consisting of:

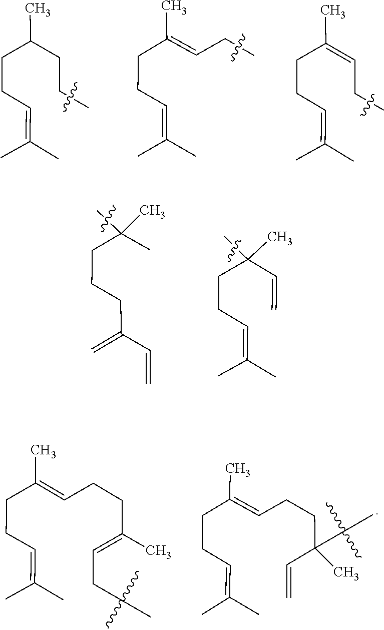

1.14 Compound 1, wherein A is selected from the group consisting of:

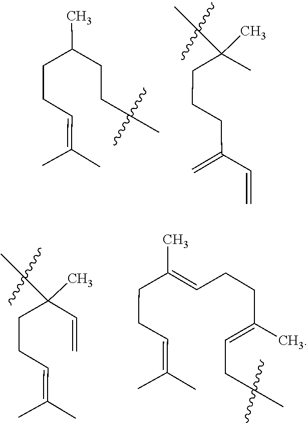 [SMILES: *CCC(C)CCC=C(C)C ; *C(C)(C)CCCC(=C)C=C ; *C(C)(C=C)CCC=C(C)C ; *C/C=C(\C)CC/C=C(\C)CCC=C(C)C]

1.15 Compound 1, wherein A is:

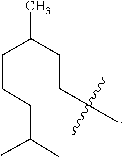 [SMILES: *CCC(C)CCCC(C)C]

1.16 Compound 1, wherein A is selected from the group consisting of:

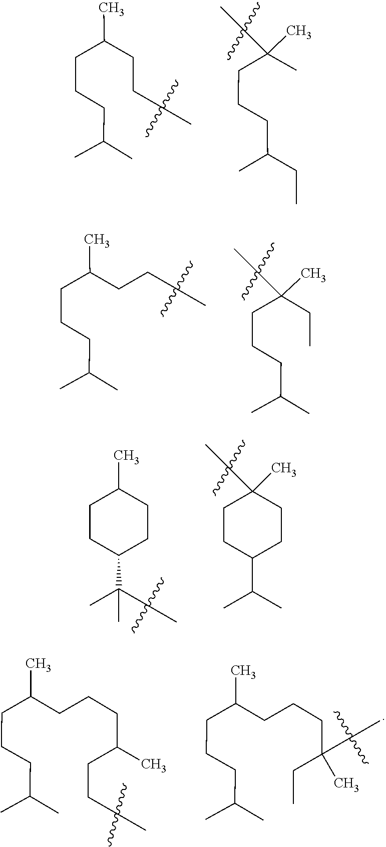

1.17 Compound 1, wherein A is selected from the group consisting of:

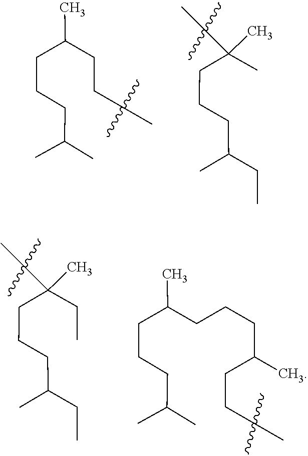 [SMILES: *CCC(C)CCCC(C)C ; *C(C)(C)CCCC(C)CC ; *C(C)(CC)CCCC(C)CC ; *CCC(C)CCCC(C)CCCC(C)C]

1.18 Compound 1, wherein A is:

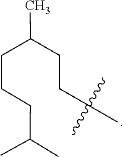 [SMILES: *CCC(C)CCCC(C)C]

1.19 Compound 1, wherein A is:

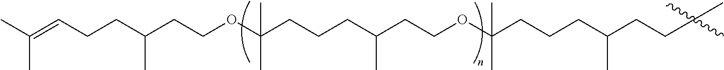

wherein n is an integer from 0-20 (e.g., 0-3, 0, 1 or 2).
1.20 Compound 1, wherein A is:

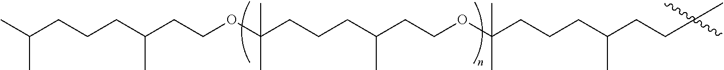

wherein n is an integer from 0-20 (e.g., 0-3, 0, 1 or 2).
1.21 Compound 1, or any of 1.1-1.20, wherein R₁ is H.
1.22 Compound 1, or any of 1.1-1.20, wherein R₁ is —COOH or —COO⁻.
1.23 Compound 1, or any of 1.1-1.20, wherein R₁ is —OH.
1.24 Compound 1, or any of 1.1-1.20, wherein R₁ and the methine carbon to which it is attached (CH—R₁) form a carbonyl (C═O);
1.25 Compound 1, or any of 1.1-1.24, wherein R₂, R₃, and R₄ are each independently selected from:
   H, C₁-C₁₂ alkyl, C₂-C₁₂ alkenyl, C₂-C₁₂ alkynyl, C₁-C₁₂ alkoxy, acyl (including C₂-C₁₂ alkylcarbonyl (—CO-alkyl) and C₆-C₂₀ arylcarbonyl (—CO-aryl)), acyloxy (—O-acyl), C₂-C₁₂ alkoxycarbonyl (—(CO)—O-alkyl), C₆-C₂₀ aryloxycarbonyl (—(CO)—O-aryl), carboxy (—COOH), carboxylato (—COO⁻), carbamoyl (—(CO)—NH₂), halo (—F, —Cl, —Br, or —I), hydroxy (—OH), and amino (—NH₂);
      wherein each of the aforementioned hydrocarbyl moieties of the preceding substituents, such as C₁-C₁₂ alkyl, C₂-C₁₂ alkenyl, C₂-C₁₂ alkynyl, and C₅-C₂₀ aryl, are each independently optionally further substituted by one or more groups selected from C₁-C₁₂ alkyl, C₁-C₁₂ alkoxy, carboxy (—COOH), carboxylato (—COOH), hydroxy (—OH), and amino (—NH₂).
1.26 Compound 1, or any of 1.1-1.24, wherein R₂, R₃, and R₄ are each independently selected from:
   H, C₁-C₁₂ alkyl, C₁-C₁₂ alkoxy, carboxy (—COOH), carboxylato (—COO⁻), hydroxy (—OH), and amino (—NH₂), wherein said C₁-C₁₂ alkyl is optionally further substituted by one or more groups selected from C₁-C₁₂ alkyl, C₁-C₁₂ alkoxy, carboxy (—COOH), carboxylato (—COO⁻), hydroxy (—OH), and amino (—NH₂).
1.27 Compound 1, or any of 1.1-1.26, wherein R₂, R₃, and R₄ are each independently selected from H, C₁-C₁₂ alkyl, carboxy (—COOH), carboxylato (—COO⁻), and hydroxy (—OH), wherein said C₁-C₁₂ alkyl is optionally further substituted by one or more groups selected from carboxy (—COOH), carboxylato (—COO⁻), and hydroxy (—OH).
1.28 Compound 1, or any of 1.1-1.27, wherein R₁ is —OH, R₂ is —OH, R₃ is H, and R₄ is —CH(OH)CH(OH)CH₂OH.
1.29 Compound 1, or any of 1.1-1.27, wherein R₁ is —H, R₂ is —COOH or —COO⁻, R₃ is —OH, and R₄ is —CH₂COOH or —CH₂COO⁻.
1.30 Compound 1, or any of 1.1-1.27, wherein R¹ is —H, R₂ is —H, R₃ is —H, and R₄ is —CH₂COOH or —CH₂COO⁻.
1.31 Compound 1, or any of 1.1-1.27, wherein R₁ is —H, R₂ is —H, R₃ is —H, and R₄ is —COOH or —COO⁻.
1.32 Compound 1, or any of 1.1-1.27, wherein R₁ and the methine carbon to which it is attached (CH—R₁) form a carbonyl (—C═O), and R₂ is —OH, R₃ is H.
1.33 Compound 1.32, wherein R₄ is —CH(OH)CH(OH)CH₂OH.
1.34 Compound 1, or any of 1.1-1.33, wherein group A is an isodecyl group, e.g., selected from 2,4-dimethyloctan-2-yl, 2,6-dimethyl-octan-1-yl, 2,6-dimethyloctan-2-yl, 3,7-dimethyloctan-1-yl, and 3,7-dimethyloctan-3-yl.
1.35 Compound 1, or any of 1.1-1.34, wherein R₁ is —OH, R₂ is —OH, R₃ is H, and R₄ is —CH(OH)CH(OH)CH₂OH, or wherein R₁ is —H, R₂ is —H, R₃ is —H, and R₄ is —CH₂COOH or —CH₂COO⁻.
1.36 Compound 1, or any of 1.1-1.35, wherein the compound is selected from the group consisting of:

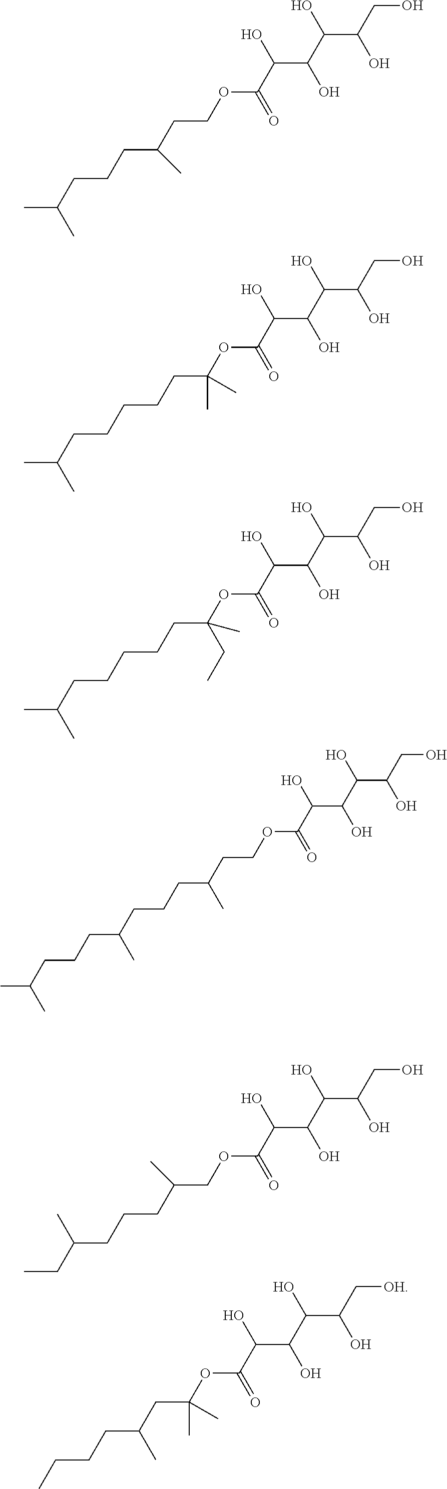 [SMILES: CC(C)CCCC(C)CCOC(=O)C(O)C(O)C(O)C(O)CO ; CC(C)CCCCCC(C)(C)OC(=O)C(O)C(O)C(O)C(O)CO ; CCC(C)(CCCCCC(C)C)OC(=O)C(O)C(O)C(O)C(O)CO ; CC(C)CCCC(C)CCCC(C)CCOC(=O)C(O)C(O)C(O)C(O)CO ; CCC(C)CCCC(C)COC(=O)C(O)C(O)C(O)C(O)CO ; CCCCC(C)CC(C)(C)OC(=O)C(O)C(O)C(O)C(O)CO]

1.37 Compound 1, or any of 1.1-1.35, wherein the compound is selected from the group consisting of:

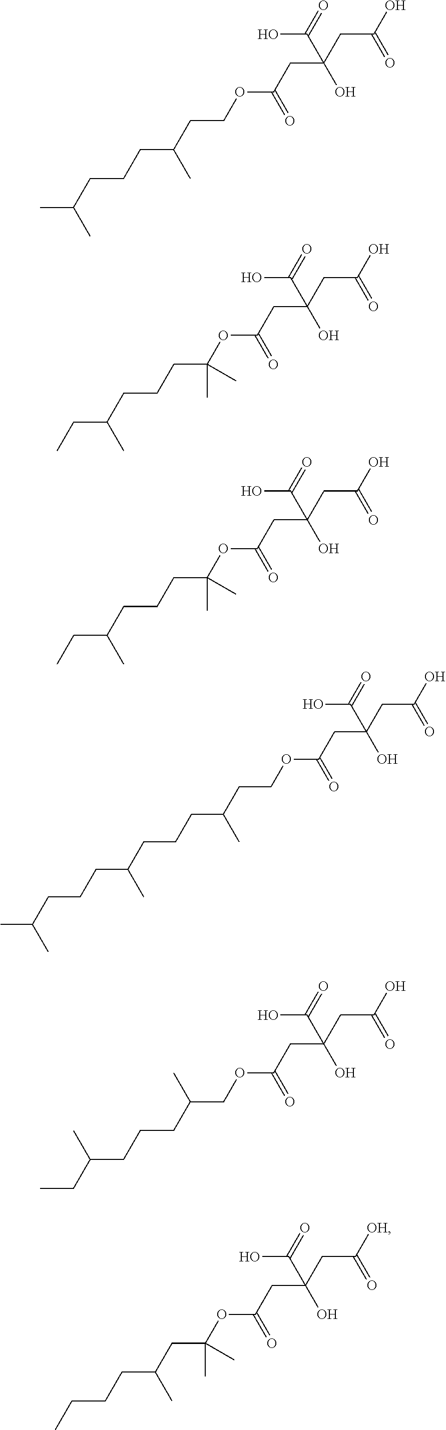 [SMILES: CC(C)CCCC(C)CCOC(=O)CC(O)(CC(=O)O)C(=O)O ; CCC(C)CCCC(C)(C)OC(=O)CC(O)(CC(=O)O)C(=O)O ; CCC(C)CCCC(C)(C)OC(=O)CC(O)(CC(=O)O)C(=O)O ; CC(C)CCCC(C)CCCC(C)CCOC(=O)CC(O)(CC(=O)O)C(=O)O ; CCC(C)CCCC(C)COC(=O)CC(O)(CC(=O)O)C(=O)O ; CCCCC(C)CC(C)(C)OC(=O)CC(O)(CC(=O)O)C(=O)O]

optionally wherein any one or more groups COOH may exist as —CH₂COO⁻.
1.38 Compound 1, or any of 1.1-1.35, wherein the compound is selected from the group consisting of:

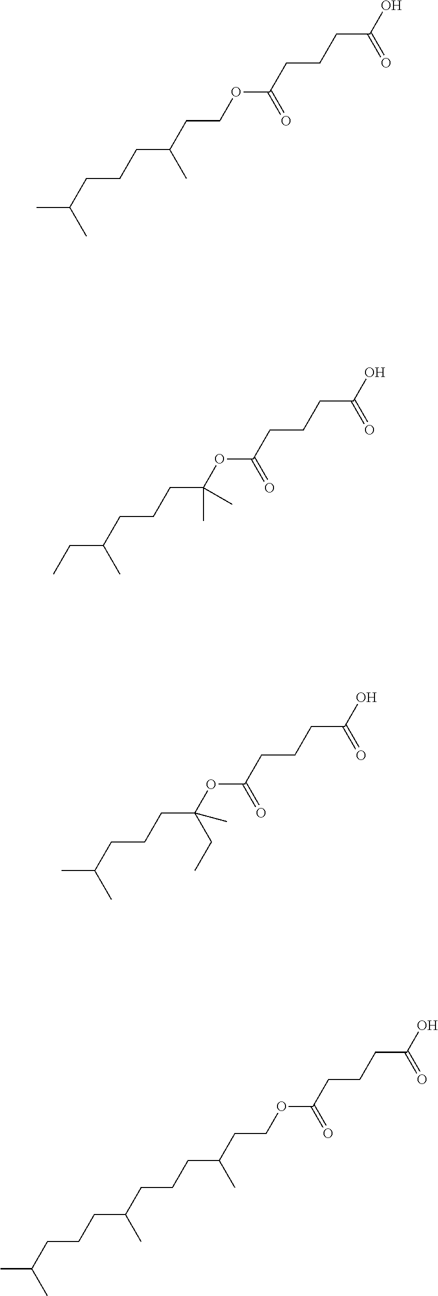 [SMILES: CC(C)CCCC(C)CCOC(=O)CCCC(=O)O ; CCC(C)CCCC(C)(C)OC(=O)CCCC(=O)O ; CCC(C)(CCCC(C)C)OC(=O)CCCC(=O)O ; CC(C)CCCC(C)CCCC(C)CCOC(=O)CCCC(=O)O]

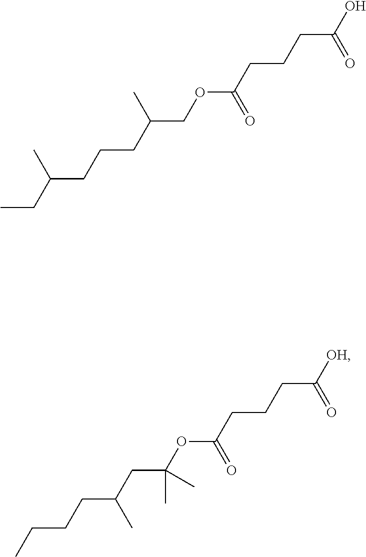 [SMILES: CCC(C)CCCC(C)COC(=O)CCCC(=O)O ; CCCCC(C)CC(C)(C)OC(=O)CCCC(=O)O]

optionally wherein any one or more groups COOH may exist as —CH₂COO⁻.
1.39 Compound 1, or any of 1.1-1.35, wherein the compound is selected from the group consisting of:

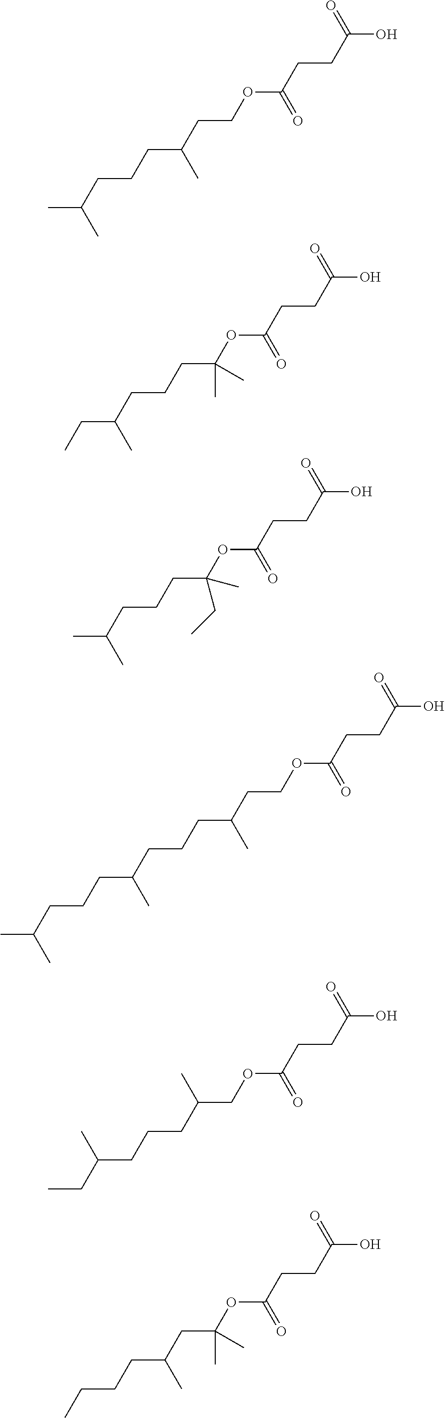 [SMILES: CC(C)CCCC(C)CCOC(=O)CCC(=O)O ; CCC(C)CCCC(C)(C)OC(=O)CCC(=O)O ; CCC(C)(CCCC(C)C)OC(=O)CCC(=O)O ; CC(C)CCCC(C)CCCC(C)CCOC(=O)CCC(=O)O ; CCC(C)CCCC(C)COC(=O)CCC(=O)O ; CCCCC(C)CC(C)(C)OC(=O)CCC(=O)O]

optionally wherein any one or more groups COOH may exist as —CH₂COO⁻.
1.40 Compound 1, or any of 1.1-1.35, wherein the compound is selected from the group consisting of:

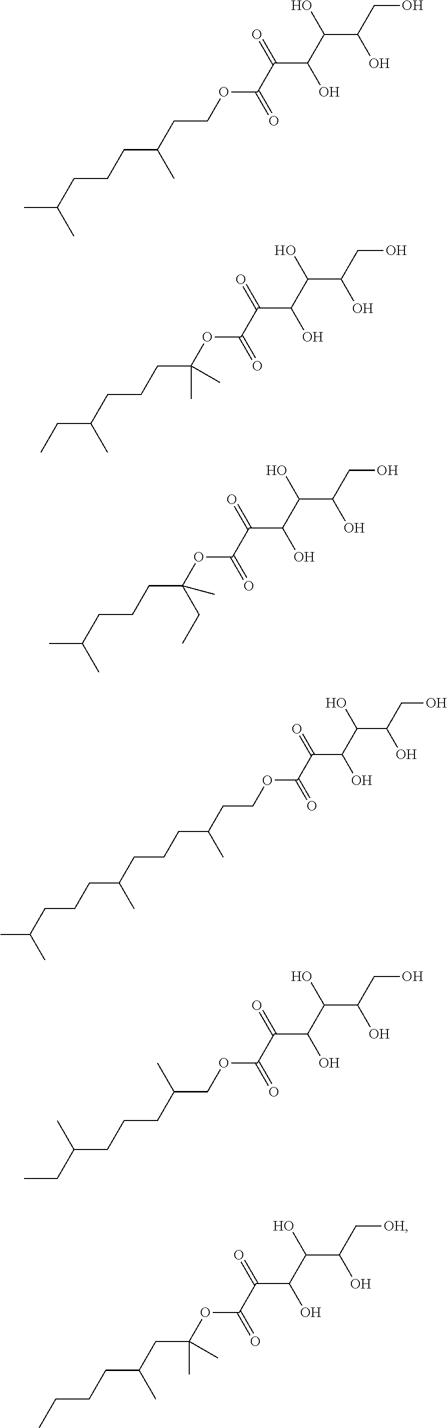 [SMILES: CC(C)CCCC(C)CCOC(=O)C(=O)C(O)C(O)C(O)CO ; CCC(C)CCCC(C)(C)OC(=O)C(=O)C(O)C(O)C(O)CO ; CCC(C)(CCCC(C)C)OC(=O)C(=O)C(O)C(O)C(O)CO ; CC(C)CCCC(C)CCCC(C)CCOC(=O)C(=O)C(O)C(O)C(O)CO ; CCC(C)CCCC(C)COC(=O)C(=O)C(O)C(O)C(O)CO ; CCCCC(C)CC(C)(C)OC(=O)C(=O)C(O)C(O)C(O)CO]

or the enol tautomers thereof,

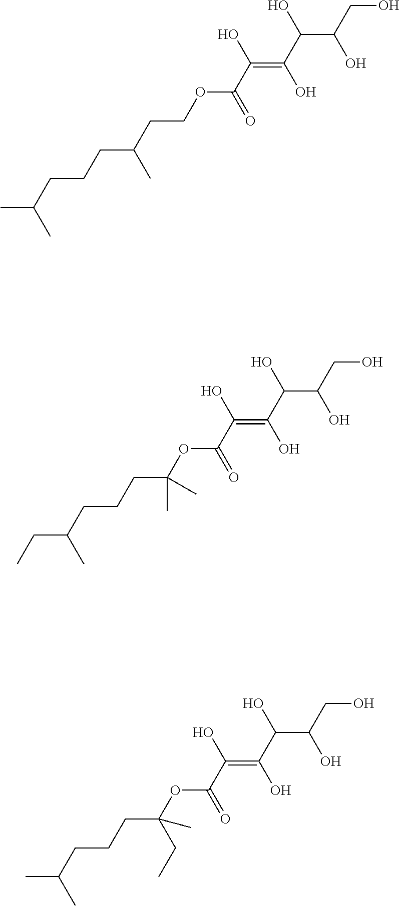 [SMILES: CC(C)CCCC(C)CCOC(=O)/C(O)=C(\O)C(O)C(O)CO ; CCC(C)CCCC(C)(C)OC(=O)/C(O)=C(\O)C(O)C(O)CO ; CCC(C)(CCCC(C)C)OC(=O)/C(O)=C(\O)C(O)C(O)CO]

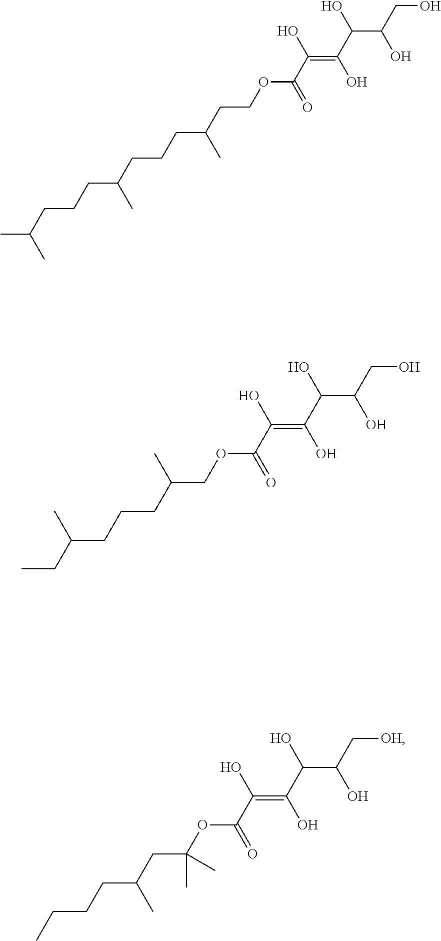 [SMILES: CC(C)CCCC(C)CCCC(C)CCOC(=O)/C(O)=C(\O)C(O)C(O)CO ; CCC(C)CCCC(C)COC(=O)/C(O)=C(\O)C(O)C(O)CO ; CCCCC(C)CC(C)(C)OC(=O)/C(O)=C(\O)C(O)C(O)CO]

whether having a cis or a trans double bond.
1.41 Any compounds 1.1-1.40, wherein the compound has a single stereogenic center within the substituent A and that center has the R configuration.
1.42 Any compounds 1.1-1.40, wherein the compound has a single stereogenic center within the substituent A and that center has the S configuration.
1.43 Any compounds 1.1-1.0, wherein the compound has two or three stereogenic centers within the substituent A and they each have the R configuration.
1.44 Any compounds 1.1-1.40, wherein the compound has two or three stereogenic centers within the substituent A and they each have the S configuration.
1.45 Compound 1, or any of 1.1-1.44, wherein the compound has a refractive index from 1.35 to 1.55, e.g., 1.40 to 1.50, or 1.42 to 1.48, or 1.43 to 1.46, or 1.44-1.45.
1.46 Compound 1, or any of 1.1-1.46, wherein the compound has a surface tension of 15 to 35 mN/m, e.g., 20 to 30 mN/m, or 22 to 28 mN/m, or 23 to 27 mN/m, or 24 to 26 mN/m, or about 25 mN/m.

The term “isodecyl” as used herein refers to any 10-carbon saturated alkyl chain that is not linear (i.e., not n-decyl).

The compounds provided by the present disclosure offer numerous improved benefits over existing compounds used for the same purpose. For example, Compound 1 et seq. provides one or more of: (a) lower melting point, (b) better lubricity, (c) better spreading (e.g., better spontaneous spreading on the skin), (d) higher refractive index, (e) better hydrolytic stability, and (f) better enzymatic stability. Without being bound by theory, it is believed that compounds as disclosed herein having an isodecyl group are provide particularly beneficial improvements over compounds of the prior art, for example, due to the increased extent of branching in the alkyl chain. Surface tension is one of the physical factors which helps provide the compounds with improved emolliency, lubricity, spreadability and “play” (i.e., feel on the skin and hair) compared to known compounds used for similar purposes. Preferably, compounds of the present disclosure have a surface tension between 15 and 35 milliNewtons/meter (mN/m). Refractive index is important from an appearance standpoint, as a higher refractive index provides for a shinier or glossier product. Preferably, compounds of the present disclosure have a refractive index between 1.35 and 1.55.

It understood that the compounds disclosed herein may include ionizable moieties, i.e., moieties which, depending on the pH of their surroundings, may exist in either an ionized (ionic) form or in a non-ionized (neutral) form. For example, carboxylic acid groups (—COOH), and amino groups (—NH₂). A carboxylic acid group may exist in a protonated form (—COOH) or in a deprotonated form (COO⁻), and the latter will generally exist as a salt with some cationic species (e.g., lithium, sodium, potassium, or other metal cations, or ammonium cations). Likewise, an amino group may exist in a deprotonated form (—NH₂) or a protonated form (—NH₃₊), and the latter will generally exist as a salt with some anionic species (e.g., halide anion). As used herein throughout, any compound having an ionizable group may have that ionizable group in either a protonated or unprotonated form, and thus, the compound may be in a neutral form or in the form of a salt with a suitable counterion.

The term “alkyl” as used herein refers to a monovalent or bivalent, branched or unbranched saturated hydrocarbon group having from 1 to 20 carbon atoms, typically although, not necessarily, containing 1 to about 12 carbon atoms, such as methyl, ethyl, n-propyl, isopropyl, n-butyl, isobutyl, t-butyl, octyl, and the like. The term alkyl also may include cycloalkyl groups. Thus, for example, the term C₆ alkyl would embrace cyclohexyl groups, the term C₅ would embrace cyclopentyl groups, the term C₄ would embrace cyclobutyl groups, and the term C₃ would embrace cyclopropyl groups. In addition, as the alkyl group may be branched or unbranched, any alkyl group of n carbon atoms would embrace a cycloalkyl group of less than n carbons substituted by additional alkyl substituents. Thus, for example, the term C₆ alkyl would also embrace methylcyclopentyl groups, or dimethylcyclobutyl or ethylcyclobutyl groups, or trimethylcyclopropyl, ethylmethylcyclopropyl or propylcyclopropyl groups.

The term “alkenyl” as used herein refers to a monovalent or bivalent, branched or unbranched, unsaturated hydrocarbon group typically although not necessarily containing 2 to about 12 carbon atoms and 1 -10 carbon-carbon double bonds, such as ethylene, n-propylene, isopropylene, n-butylene, isobutylene, t-butylene, octylene, and the like. In like manner as for the term “alkyl”, the term “alkenyl” also embraces cycloalkenyl groups, both branched an unbranched with the double bond optionally intracyclic or exocyclic.

The term “alkynyl” as used herein refers to a monovalent or bivalent, branched or unbranched, unsaturated hydrocarbon group typically although not necessarily containing 2 to about 12 carbon atoms and 1-8 carbon-carbon triple bonds, such as ethyne, propyne, butyne, pentyne, hexyne, heptyne, octyne, and the like. In like manner as for the term “alkyl”, the term “alkynyl” also embraces cycloalkynyl groups, both branched an unbranched, with the triple bond optionally intracyclic or exocyclic.

The term “aryl” as used herein refers to an aromatic hydrocarbon moiety comprising at least one aromatic ring of 5-6 carbon atoms, including, for example, an aromatic hydrocarbon having two fused rings and 10 carbon atoms (i.e., a naphthalene).

By “substituted” as in “substituted alkyl,” “substituted alkenyl,” “substituted alkynyl,” and the like, it is meant that in the alkyl, alkenyl, alkynyl, or other moiety, at least one hydrogen atom bound to a carbon atom is replaced with one or more non-hydrogen substituents, e.g., by a functional group.

The terms “branched” and “linear” (or “unbranched”) when used in reference to, for example, an alkyl moiety of Cₐ to C_{b} carbon atoms, applies to those carbon atoms defining the alkyl moiety. For example, for a C₄ alkyl moiety, a branched embodiment thereof would include an isobutyl, whereas an unbranched embodiment thereof would be an n-butyl. However, an isobutyl would also qualify as a linear C₃ alkyl moiety (a propyl) itself substituted by a C₁ alkyl (a methyl).

Unless otherwise specified, any carbon atom with an open valence may be substituted by an additional functional group. Examples of functional groups include, without limitation: halo, hydroxyl, sulfhydryl, C₁-C₂₀ alkoxy, C₂-C₂₀ alkenyloxy, C₂-C₂₀ alkynyloxy, C₅-C₂₀ aryloxy, acyl (including C₂-C₂₀ alkylcarbonyl (—CO-alkyl) and C₆-C₂₀ arylcarbonyl (—CO-aryl)), acyloxy (—O-acyl), C₂-C₂₀ alkoxycarbonyl (—(CO)—O-alkyl), C₆-C₂₀ aryloxycarbonyl (—(CO)—O-aryl), halocarbonyl (—CO)—X where X is halo), C₂-C₂₀ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-substituted C₁-C₂₀ alkylcarbamoyl (—(CO)—NH(C₁-C₂₀ alkyl)), di-substituted alkylcarbamoyl (—(CO)—N(C₁-C₂₀ alkyl)₂), mono-substituted arylcarbamoyl (—(CO)—NH-aryl), thiocarbamoyl (—(CS)—NH₂), carbamido (—NH—(CO)—NH₂), cyano (—CN), isocyano (—N⁺≡C⁻), cyanato (—O—≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁-C₂₀ alkyl)-substituted amino, mono- and di-(C₅-C₂₀ aryl)-substituted amino, C₂-C₂₀ alkylamido (—NH—(CO)-alkyl), C₅-C₂₀ arylamido (—NH—(CO)-aryl), imino (—CR═NH where R=hydrogen, C₁-C20 alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O⁻), C₁-C₂₀ alkylsulfanyl (—S-alkyl; also termed “alkylthio”), arylsulfonyl (—S-aryl; also termed “arylthio”), C₁-C₂₀ alkylsulfinyl (—(SO)—alkyl), C₅-C₂₀ arylsulfinyl (—(SO)-aryl), C₁-C₂₀ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O⁻)), phospho (—PO₂),-phosphino (—PH₂), mono- and di-(C₁-C₂₀ alkyl)-substituted phosphino, mono- and di-(C₅-C₂₀ aryl)-substituted phosphino; and the hydrocarbyl moieties such as C₁-C20 alkyl (including C₁-C₁₈ alkyl, further including C₁-C₁₂ alkyl, and further including C₁-C₆ alkyl), C₂-C₂₀ alkenyl (including C₂-C₁₈ alkenyl, further including C₂-C₁₂ alkenyl, and further including C₂-C₆ alkenyl), C₂-C₂₀ alkynyl (including C₂-C₁₈ alkynyl, further including C₂-C₁₂ alkynyl, and further including C₂-C₆ alkynyl), C₅-C₃₀ aryl (including C₅-C₂₀ aryl, and further including C₅-C₁₂ aryl), and C₆-C₂₀ aralkyl (including C₆-C₂₀ aralkyl, and further including C₆-C₁₂ aralkyl). In addition, the aforementioned functional groups may, if a particular group permits, be further substituted with one or more additional functional groups or with one or more hydrocarbyl moieties such as those specifically enumerated above. For example, the alkyl or alkenyl group may be branched. For example, the “substituent” is an alkyl group, e.g., a methyl group.

In a second aspect, the present disclosure provides a method of making the Compound 1, et seq., comprising the step of reacting a compound of the Formula A, with a compound of Formula B, or an ester, activated ester or acyl halide thereof, in a condensation reaction to form the compound of Formula I:

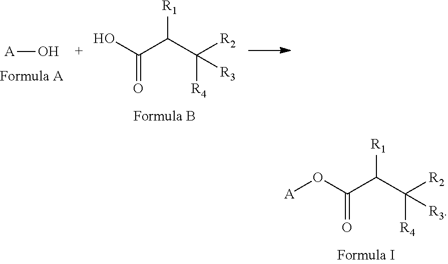

wherein substituents A, R₁, R₂, R₃, and R₄, are as defined hereinabove. In some embodiments, the reaction is conducted by reacting the compound of Formula A and the compound of Formula B in the presence of an acid catalyst, optionally under dehydrating conditions. Preferably, the acid catalyst is selected from sulfuric acid, hydrochloric acid, phosphoric acid, toluenesulfonic acid, methanesulfonic acid, or an acidic ion exchange resin, such as an Amberlyst-type resin. In some embodiments, the reaction further comprises a dehydrating agent, such as sodium sulfate, magnesium sulfate, phosphorus pentoxide, or the like. In a preferred embodiment, the reaction comprises a mixture of sulfuric acid and magnesium sulfate, optionally in a hydrocarbon solvent, such as heptane. In some embodiments, the magnesium sulfate is first suspended in a hydrocarbon solvent, such as heptane, and concentration sulfuric acid is added to form, after removal of the solvent, a solid MgSO₄/H₂SO₄ adduct which can be used directly as an acidic catalyst for the condensation reaction. Preferably, this solid adduct is added directly to the neat reaction components (e.g., where the terpene alcohol of Formula A and/or the acid of Formula B is a liquid). In some embodiments, the reaction is conducted by reacting the compound of Formula A and the compound of Formula B in the presence of a coupling reagent, for example, 1,1-carbonyl-di-imidazole. In some embodiments, the reaction is conducted by reacting the compound of Formula A with an activated derivative of the compound of Formula B, such as an acyl halide or acid anhydride of the compound of Formula B. In some embodiments, the reaction is conducted under basic conditions, e.g., by reacting a compound of Formula A with a compound of Formula B, or an ester, activated ester, or acyl halide thereof, in the presence of a base (e.g., a hydroxide base, an alkoxide base, a carbonate base, a bicarbonate base, a hydride base, an organometallic base, or an amide base). In some embodiments, the reaction is conducted by reacting a salt compound of Formula A, such as a lithium salt, a sodium salt, or a potassium salt, with a compound of Formula B, or an ester, activated ester, or acyl halide thereof. In some embodiments said salt is formed in-situ. Suitable bases include sodium hydroxide, sodium methoxide, sodium ethoxide, sodium propoxide, sodium isopropoxide, sodium butoxide, sodium tert-butoxide, sodium carbonate, sodium bicarbonate, sodium hydride, sodium amide, potassium hydroxide, potassium methoxide, potassium ethoxide, potassium propoxide, potassium isopropoxide, potassium tert-butoxide, potassium carbonate, potassium bicarbonate, potassium hydride, potassium amide, lithium hydroxide, lithium methoxide, lithium tert-butoxide, lithium carbonate, lithium amide, lithium diisopropylamide, lithium hexamethyldisilazide, lithium tetramethylpiperidide, n-butyllithium, s-butyllithium, and t-butyllithium.

Suitable solvents and reactions conditions (concentration, time, temperature) for the conducting the reactions are generally known to those skilled in the art and are not limited in any way in the present disclosure. Depending on the choice of reagents, suitable solvents may include one or more of apolar, polar protic and/or polar aprotic solvents, for example hydrocarbons, ethers, and esters.

In some embodiments, the reaction is carried out at a temperature of −25° C. to 200° C. In a preferred embodiment, the reaction is run at 25 to 150° C., or 50 to 100° C. In some embodiments, the reaction is carried out for 0.1 to 100 hours. In a preferred embodiment the reaction is run for 0.5-12 hours, or 0.5 to 6 hours, or 1 to 3 hours.

The compound Formula A, used to make the Compound 1 et seq. of the present disclosure, is a terpene alcohol or a derivative thereof (e.g., a hydrogenated terpene alcohol). Preferably the terpene alcohol is obtained from or isolated from a natural renewable resource. For example, the each of the following terpene alcohols can be obtained by extraction from numerous plant species: citronellol, isocitronellol, geraniol, nerol, menthol, myrcenol, linalool, thymol, α-terpineol, β-terpineol, γ-terpineol, borneol, farnesol, nerolidol, and carotol. The essential oils of many trees and plants, such as rose oil, palmarosa oil, citronella oil, lavender oil, coriander oil, thyme oil, peppermint oil, and pine oil, have significant amounts of these terpene alcohols.

In a preferred embodiment, however, the terpene alcohols may be derived semi-synthetically (e.g., by double bond hydration reactions) from naturally derived terpenes. Terpenes are much more abundant in nature than the corresponding terpene alcohols. Common terpenes include: alpha-pinene, beta-pinene, alpha-terpinene, beta-terpinene, gamma-terpinene, delta-terpinene (terpinolene), myrcene, limonene, camphene, carene, sabinene, alpha-ocimene, beta-ocimene, alpha-thujene, and beta-thujene. Alpha-pinene is the most abundant naturally occurring terpene in nature, being present in a high concentration in various tree resins and oils, such as pine oil and oleoresin (and its derivative turpentine). Numerous terpene oils can be derived from the terpenes present in turpentine, pine oil, and similar materials. Turpentine is a major by-product of the paper and pulp industries, so using this material as a source for terpene alcohols would be both economical and environmentally friendly.

In addition, the terpene alcohols can be prepared semi-synthetically from either isobutylene, isoprenol, or ethanol. Ethanol, as well as methanol and tert-butanol, can be derived in large volumes from the fermentation of biorenewable sugars, such as from corn, cane sugar or beet sugar. Isobutylene can be derived from tert-butanol by elimination or from ethanol by mixed oxidation to acetaldehyde and acetone and aldol condensation, and isoprenol can be derived from isobutylene by reaction with formaldehyde, and formaldehyde can be made by oxidation of methanol. Methanol and ethanol can also be derived from the by-product fractions from commercial ethanol distillation (e.g., in the making of spirits). By these routes, the Compounds of the present disclosure can all be made entirely from biorenewable resources such as trees and plants.

Thus, in some embodiments of the present disclosure, the Method of making Compound 1 et seq. may further comprise one or more of the following steps: (1) harvesting of one or more crops or grains (e.g., corn, beets, sugarcane, barley, wheat, rye, or sorghum), (2) fermenting such harvested crops or grains, (3) obtaining from such fermentation one or more C₁₋₄ aliphatic alcohols (e.g., methanol, ethanol, isobutanol, tert-butanol, or any combination thereof), (4) converting said alcohols to isobutylene and/or isoprenol, (5) converting said isobutylene or isoprenol to one or more terpenes (e.g., alpha-pinene, beta-pinene, alpha-terpinene, beta-terpinene, gamma-terpinene, delta-terpinene (terpinolene), myrcene, limonene, camphene, carene, sabinene, alpha-ocimene, beta-ocimene, alpha-thujene, and beta-thujene); (6) extracting or isolating one or more terpenes from naturally occurring plant and tree extracts, such as essential oils and resins (e.g., rosin, dammars, mastic, sandarac, frankincense, elemi, turpentine, copaiba, oleoresin, pine oil, cannabis oil, coriander oil), and (7) converting such terpenes to one or more terpene alcohols (e.g., citronellol, isocitronellol, geraniol, nerol, menthol, myrcenol, linalool, thymol, α-terpineol, β-terpineol, γ-terpineol, borneol, farnesol, nerolidol, and carotol).

The skilled person will recognize that in some embodiments, the compound of Formula B may be an alpha-keto acid, such as pyruvic acid, oxaloacetic acid, alpha-ketoglutaric acid, or 2-ketogluconic acid. The product of Formula I, therefore, is thus an alpha-keto ester, such as a pyruvate, oxaloacetate or alpha-ketoglutarate. In such situations, the compound of Formula B and/or the compound of Formula I may exist in either a keto- or enol-tautomeric form. Moreover, the skilled person will recognize that ascorbic acid is a lactone of an alpha-keto acid, and therefore, the compound of Formula B may be ascorbic acid, and the resulting product Compound 1 may be an alpha-keto ester. For example, in some embodiments, the present disclosure would provide as follows:

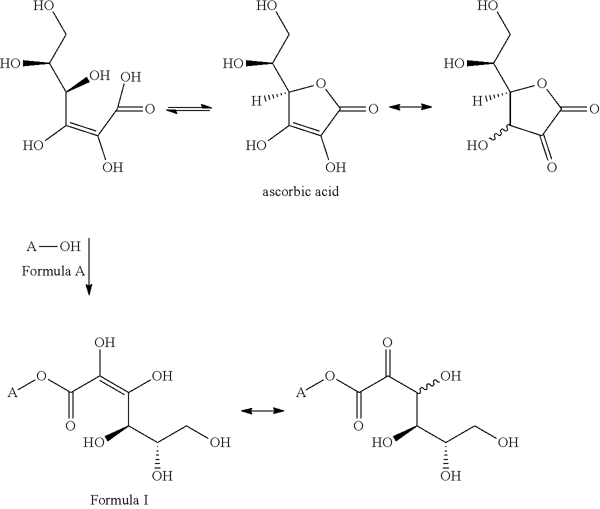

In another aspect, the present disclosure provides a composition comprising Compound 1 or any of 1.1 to 1.46, optionally in admixture with one or more pharmaceutically acceptable, cosmetically acceptable, or industrially acceptable excipients or carriers, for example, solvents, oils, surfactants, emollients, diluents, glidants, abrasives, humectants, polymers, plasticizer, catalyst, antioxidant, coloring agent, flavoring agent, fragrance agent, antiperspirant agent, antibacterial agent, antifungal agent, hydrocarbon, stabilizer, or viscosity controlling agent. In some embodiments, the composition is a pharmaceutical composition, or a cosmetic composition, or a sunscreen composition, or a plastic composition, or a lubricant composition, or a personal care composition (e.g., a soap, skin cream or lotion, balm, shampoo, body wash, hydrating cream, deodorant, antiperspirant, after-shave lotion, cologne, perfume, or other hair care or skin care product), or a cleaning composition (e.g., a surface cleaner, a metal cleaner, a wood cleaner, a glass cleaner, a body cleaner such as a soap, a dish-washing detergent, or a laundry detergent), or an air freshener.

In preferred embodiments, such Compositions comprise a Compound according to the present disclosure having an isodecyl group. In a particularly preferred embodiment, such Compositions also comprise another excipient having a decyl or isodecyl group, such as, decyl or isodecyl alcohol, decanoic or isodecanoic acids, decyl or isodecyl ethers, or decyl or isodecyl esters. For example, such Compositions may comprise a combination of one or more of the isodecyl compounds of Examples 1 to 8.

The compounds of the present disclosure, e.g., Compound 1, et seq., may be used with, e.g.: perfumes, soaps, insect repellants and insecticides, detergents, household cleaning agents, air fresheners, room sprays, pomanders, candles, cosmetics, toilet waters, pre- and aftershave lotions, talcum powders, hair-care products, body deodorants, anti-perspirants, shampoo, cologne, shower gel, hair spray, and pet litter.

Fragrance and ingredients and mixtures of fragrance ingredients that may be used in combination with the disclosed compound for the manufacture of fragrance compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products.

In some embodiments, the present disclosure provides personal care compositions including, but not limited to, soaps (liquid or solid), body washes, skin and hair cleansers, skin creams and lotions (e.g., facial creams and lotions, face oils, eye cream, other anti-wrinkle products), ointments, sunscreens, moisturizers, hair shampoos and/or conditioners, deodorants, antiperspirants, other conditioning products for the hair, skin, and nails (e.g., shampoos, conditioners, hair sprays, hair styling gel, hair mousse), decorative cosmetics (e.g., nail polish, eye liner, mascara, lipstick, foundation, concealer, blush, bronzer, eye shadow, lip liner, lip balm,) and dermocosmetics.

In some embodiments, the personal care compositions may include organically-sourced ingredients, vegan ingredients, gluten-free ingredients, environmentally-friendly ingredients, natural ingredients (e.g. soy oil, beeswax, rosemary oil, vitamin E, coconut oil, herbal oils etc.), comedogenic ingredients, natural occlusive plant based ingredients (e.g. cocoa, shea, mango butter), non-comedogenic ingredients, bakuchiol (a plant derived compound used as a less-irritating, natural alternative to retinol), color active ingredients (e.g., pigments and dyes); therapeutically-active ingredients (e.g., vitamins, alpha hydroxy acids, corticosteroids, amino acids, collagen, retinoids, antimicrobial compounds), sunscreen ingredients and/or UV absorbing compounds, reflective compounds, oils (such as castor oil and olive oil, or high-viscosity oils), film formers, high molecular weight esters, antiperspirant active ingredients, glycol solutions, water, alcohols, emulsifiers, gellants, emollients, water, polymers, hydrocarbons, conditioning agents, and/or aliphatic esters.

In some embodiments, the present compositions are gluten free.

In some embodiments, the present compositions are formulated as oil-in-water emulsions, or as water-in-oil emulsions. In some embodiments, the compositions may further comprise one or more hydrocarbons, such as heptane, octane, nonane, decane, undecane, dodecane, isododecane, tridecane, tetradecane, pentadecane, hexadecane, heptadecane, octadecane, nonadecane, henicosane, docosane, and tricosane, and any saturated linear or saturated branched isomer thereof.

As used herein, the phrases “for example,” “for instance,” “such as,” or “including” are meant to introduce examples that further clarify more general subject matter. These examples are provided only as an aid for understanding the disclosure, and are not meant to be limiting in any fashion. Furthermore, as used herein, the terms “may,” “optional,” “optionally,” or “may optionally” mean that the subsequently described circumstance may or may not occur, so that the description includes instances where the circumstance occurs and instances where it does not. For example, the phrase “optionally present” means that an object may or may not be present, and, thus, the description includes instances wherein the object is present and instances wherein the object is not present.

As used herein, the phrase “having the formula” or “having the structure” is not intended to be limiting and is used in the same way that the term “comprising” is commonly used.

In the present specification, the structural formula of the compounds represents a certain isomer for convenience in some cases, but the present invention includes ail isomers, such as geometrical isomers, optical isomers based on an asymmetrical carbon, stereoisomers, tautomers, and the like. In addition, a crystal polymorphism may be present for the compounds represented by the formulas describe herein. It is noted that any crystal form, crystal form mixture, or anhydride or hydrate thereof is included in the scope of the present invention.

“Tautomers” refers to compounds whose structures differ markedly in arrangement of atoms, but which exist in easy and rapid equilibrium. It is to be understood that the compounds of the invention may be depicted as different tautomers. it should also be understood that when compounds have tautomeric forms, ail tautomeric forms are intended to be within the scope of the invention, and the naming of the compounds does not exclude any tautomeric form. Further, even though one tautomer may be described, the present invention includes all tautomers of the present compounds.

As used herein, the term “salt” can include acid addition salts including hydrochlorides, hydrobromides, phosphates, sulfates, hydrogen sulfates, alkylsulfonates, arylsulfonates, acetates, benzoates, citrates, maleates, fumarates, succinates, lactates, and tartrates; alkali metal cations such as Na+, K+, Li+, alkali earth metal salts such as Mg2+ or Ca2+, or organic amine salts, or organic phosphonium salts.

All percentages used herein, unless otherwise indicated, are by volume.

All ratios used herein, unless otherwise indicated, are by molarity.

Although specific embodiments of the present disclosure have been described with reference to the preparations and schemes, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### EXAMPLES

Having been generally described herein, the follow non-limiting examples are provided to further illustrate this invention.

The compounds disclosed herein can be prepared through a number of straightforward esterification or transesterification processes. One preferred method involves the use of combinations of MgSO₄ and H₂O₄, in a similar vein to that of Wright, et al. in Tetrahedron Letters, Vol. 38, No. 42, pp. 7345-7348, 1997. In an even more preferred method, however, the MgSO₄/H₂SO₄ catalyst is prepared in advance from a non-polar organic solvent such as heptane.

In this approach the MgSO₄ is suspended in solution with stirring under inert atmosphere, (e.g., 10 g of MgSO₄ in 40 g of heptane), and concentrated H₂SO₄ is added dropwise to the solution. The mixture is stirred for some time, e.g., 15 minutes or 1 hour, and the heptane phase is then filtered off, leaving a white solid powder that can be further dried under vacuum or blown dry with inert air, e.g., nitrogen or argon. This white solid can then be used as a powerful esterification catalyst that is especially preferred for making tertiary esters from tertiary alcohols and/or suitably substituted olefins.

### Example 1. Isodecyl Citrate (2,6-dimethyloctan-1-yl citrate)

2,6-Dimethyloctanol (1 equivalent) is combined with citric acid (1 equivalent) and 50 grams of the MgSO₄/H₂SO₄ solid catalyst per kilogram of alcohol under an inert atmosphere in a 5-liter glass reactor vessel. The solution is then stirred for 8 hours at 80° C. with nitrogen bubbling. The gas outlet of the glass reactor is attached to a condenser to condense and collect excess methanol. The reaction is then brought to room temperature, and then 100 grams of potassium carbonate is slowly added to the solution. It is then stirred for 2 hours and filtered. Excess 2,6-dimethyloctanol is removed under reduced pressure and the desired product is further isolated by distillation.

### Example 2. Isodecyl Glutarate (2,4-dimethyloctan-2-yl glutarate)

2,4-Dimethyloctan-2-ol (1 equivalent) is combined with glutaric acid (1 equivalent) and 50 grams of the MgSO₄/H₂SO₄ solid catalyst per kilogram of alcohol under an inert atmosphere in a 5-liter glass reactor vessel. The solution is then stirred for 8 hours at 100° C. with nitrogen bubbling. The gas outlet of the glass reactor is attached to a condenser to condense and collect excess water. The reaction is then brought to room temperature, and then 400 grams of potassium carbonate is slowly added to the solution. It is then stirred for 2 hours and filtered. Excess 2,4-dimethyloctan-2-ol is removed under reduced pressure and the desired product is further isolated by distillation.

### Example 3. Isodecyl Citrate (3,7-dimethyloctan-1-yl citrate)

3,7-Dimethyl-1-octanol (a.k.a. dihydrocitronellol or tetrahydrogeraniol) (1 equivalent) is combined with citric acid (1 equivalent) and 50 grams of the MgSO₄/H₂SO₄ solid catalyst per kilogram of alcohol under an inert atmosphere in a 5-liter glass reactor vessel. The solution is then stirred for 8 hours at 80° C. with nitrogen bubbling. The gas outlet of the glass reactor is attached to a condenser to condense and collect excess methanol. The reaction is then brought to room temperature, and then 100 grams of potassium carbonate is slowly added to the solution. It is then stirred for 2 hours and filtered. Excess 3,7-dimethyl-1-octanol is removed under reduced pressure and the desired product is further isolated by distillation.

### Example 4. Isodecyl Glutarate (3,7-dimethyloctan-1-yl glutarate)

3,7-Dimethyl-1-octanol (1 equivalent) is combined with glutaric acid (1 equivalent) and 50 grams of the MgSO₄/H₂SO₄ solid catalyst per kilogram of alcohol under an inert atmosphere in a 5-liter glass reactor vessel. The solution is then stirred for 8 hours at 100° C. with nitrogen bubbling. The gas outlet of the glass reactor is attached to a condenser to condense and collect excess water. The reaction is then brought to room temperature, and then 400 grams of potassium carbonate is slowly added to the solution. It is then stirred for 2 hours and filtered. Excess 3,7-dimethyl-1-octanol is removed under reduced pressure and the desired product is further isolated by distillation.

### Example 5. Isodecyl Citrate (2,6-dimethyloctan-2-yl citrate)

2,6-Dimethyl-2-octanol (a.k.a. tetrahydromyrcenol) (1 equivalent) is combined with citric acid (1 equivalent) and 50 grams of the MgSO₄/H₂SO₄ solid catalyst per kilogram of alcohol under an inert atmosphere in a 5-liter glass reactor vessel. The solution is then stirred for 8 hours at 80° C. with nitrogen bubbling. The gas outlet of the glass reactor is attached to a condenser to condense and collect excess methanol. The reaction is then brought to room temperature, and then 100 grams of potassium carbonate is slowly added to the solution. It is then stirred for 2 hours and filtered. Excess 2,6-dimethyl-2-octanol is removed under reduced pressure and the desired product is further isolated by distillation.

### Example 6. Isodecyl Glutarate (2,6-dimethyloctan-2-yl glutarate)

2,6-Dimethyl-2-octanol (1 equivalent) is combined with glutaric acid (1 equivalent) and 50 grams of the MgSO₄/H₂SO₄ solid catalyst per kilogram of alcohol under an inert atmosphere in a 5-liter glass reactor vessel. The solution is then stirred for 8 hours at 100° C. with nitrogen bubbling. The gas outlet of the glass reactor is attached to a condenser to condense and collect excess water. The reaction is then brought to room temperature, and then 400 grams of potassium carbonate is slowly added to the solution. It is then stirred for 2 hours and filtered. Excess 2,6-dimethyl-2-octanol is removed under reduced pressure and the desired product is further isolated by distillation.

### Example 7. Isodecyl Citrate (3,7-dimethyloctan-3-yl citrate)

3,7-Dimethyl-3-octanol (a.k.a. tetrahydrolinalool) (1 equivalent) is combined with citric acid (1 equivalent) and 50 grams of the MgSO₄/H₂SO₄ solid catalyst per kilogram of alcohol under an inert atmosphere in a 5-liter glass reactor vessel. The solution is then stirred for 8 hours at 80° C. with nitrogen bubbling. The gas outlet of the glass reactor is attached to a condenser to condense and collect excess methanol. The reaction is then brought to room temperature, and then 100 grams of potassium carbonate is slowly added to the solution. It is then stirred for 2 hours and filtered. Excess 3,7-dimethyl-3-octanol is removed under reduced pressure and the desired product is further isolated by distillation.

### Example 8. Isodecyl Glutarate (3,7-dimethyloctan-3-yl glutarate)

3,7-Dimethyl-3-octanol (1 equivalent) is combined with glutaric acid (1 equivalent) and 50 grams of the MgSO₄/H₂SO₄ solid catalyst per kilogram of alcohol under an inert atmosphere in a 5-liter glass reactor vessel. The solution is then stirred for 8 hours at 100° C. with nitrogen bubbling. The gas outlet of the glass reactor is attached to a condenser to condense and collect excess water. The reaction is then brought to room temperature, and then 400 grams of potassium carbonate is slowly added to the solution. It is then stirred for 2 hours and filtered. Excess 3,7-dimethyl-3-octanol is removed under reduced pressure and the desired product is further isolated by distillation.

The compounds of the above Examples are believed to offer numerous improved benefits over existing compounds used for the same purpose. For example, these compounds may provide one or more of: (a) lower melting point, (b) better lubricity, (c) better spreading (e.g., better spontaneous spreading on the skin), (d) higher refractive index, (e) better hydrolytic stability, and (f) better enzymatic stability.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

We claim:

1. A terpene alcohol ester surfactant compound of the general formula (I): 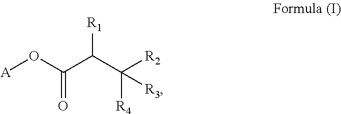
in free or salt form, wherein A is the core of a terpene alcohol or derivative thereof, and wherein R₁ is selected from hydrogen, carboxy (—COOH), carboxylato (—COO⁻), and hydroxy (—OH), or wherein R₁ and the methine carbon to which it is attached (CH—R₁) form a carbonyl (—C═O); and wherein R₂, R₃, and R₄ are each independently selected from:
H, C₁-C₁₂ alkyl, C₂-C₁₂ alkenyl, C₂-C₁₂ alkynyl, C₁-C₁₂ alkoxy, C₂-C₁₂ alkenyloxy, C₂-C₁₂ alkynyloxy, C₅-C₂₀ aryloxy, acyl (including C₂-C₁₂ alkylcarbonyl (—CO-alkyl) and C₆-C₂₀ arylcarbonyl (—CO-aryl)), acyloxy (—O-acyl), C₂-C₁₂ alkoxycarbonyl (—(CO)—O-alkyl), C₆-C₂₀ aryloxycarbonyl (—(CO)—O-aryl), C₂-C₁₂ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-N-substituted C₁-C₁₂ alkylcarbamoyl (—(CO)—NH(C₁-C₁₂ alkyl)), di-N-substituted alkylcarbamoyl (—(CO)—N(C₁-C₁₂ alkyl)₂), mono-N-substituted arylcarbamoyl (—(CO)—NH-aryl), halo (—F, —Cl, —Br, or —I), hydroxy (—OH), cyano (—C≡N), amino (—NH₂), mono- and di-N—(C₁-C₁₂ alkyl)-substituted amino, mono- and di-N—(C₅-C₂₀ aryl)-substituted amino, C₂-C₁₂ alkylamido (—NH—(CO)-alkyl), C₅-C₂₀ arylamido (—NH—(CO)-aryl), imino (—CR^{a}═NH where R^{a} is selected from hydrogen, C₁-C₁₂ alkyl, C₅-C₂₀ aryl, C₆-C₂₀ alkaryl, C₆-C₂₀ aralkyl, etc.), alkylimino (—CR^{b}═N(alkyl), wherein R^{b} is selected from hydrogen, alkyl, aryl, alkaryl, etc.), arylimino (—CR^{c}═N(aryl), where R^{c} is selected from hydrogen, alkyl, aryl, alkaryl, etc.), nitro (—NO₂), C₁-C₁₂ alkylsulfonyl (—SO₂-alkyl), and C₅-C₂₀ arylsulfonyl (—SO₂-aryl);
wherein each of the aforementioned hydrocarbyl moieties of the preceding substituents, such as C₁-C₁₂ alkyl, C₂-C₁₂ alkenyl, C₂-C₁₂ alkynyl, and C₅-C₂₀ aryl, are each independently optionally further substituted by one or more groups selected from C₁-C₁₂ alkyl, C₂-C₁₂ alkenyl, C₂-C₁₂ alkynyl, C₁-C₁₂ alkoxy, C₂-C₁₂ alkenyloxy, C₂-C₁₂ alkynyloxy, C₅-C₂₀ aryloxy, acyl (including C₂-C₁₂ alkylcarbonyl (—CO-alkyl) and C₆-C₂₀ arylcarbonyl (—CO-aryl)), acyloxy (—O-acyl), C₂-C₁₂ alkoxycarbonyl (—(CO)—O-alkyl), C₆-C₂₀ aryloxycarbonyl (—(CO)—O-aryl), C₂-C₁₂ alkylcarbonato (—O—(CO)—O-alkyl), C₆-C₂₀ arylcarbonato (—O—(CO)—O-aryl), carboxy (—COOH), carboxylato (—COO⁻), halo (—F, —Cl, —Br, or —I), hydroxy (—OH), cyano (—C≡N), and amino (—NH₂)
provided that, at least one of R₁, R₂, R₃, or R₄ is a polar or ionic group (e.g., at least one of R₁, R₂, or R₃ is —OH, —COO⁻, or —COOH, or at least R₄ is or comprises a group selected from —OH, —COOH, —NH₂, or —NO₂.

2. The compound of claim 1, wherein A is the core of a terpene alcohol, or derivative thereof, wherein said terpene is a monoterpene, sesquiterpene, diterpene, sesterterpene, or triterpene.

3. The compound of claim 1, wherein A is the core of a terpene alcohol, or derivative thereof, wherein said terpene alcohol is selected from citronellol, isocitronellol, geraniol, nerol, menthol, myrcenol, linalool, thymol, a-terpineol, b-terpineol, g-terpineol, borneol, farnesol, nerolidol, and carotol.

4. The compound of claim 3, wherein said terpene alcohol is selected from citronellol, myrcenol, linalool, and farnesol.

5. The compound of claim 1, wherein said terpene alcohol, or derivative thereof, is fully saturated (e.g., said terpene alcohol is a fully saturated monoterpene derivative, e.g., an isodecyl moiety).

6. The compound of claim 1, wherein A is selected from the group consisting of:

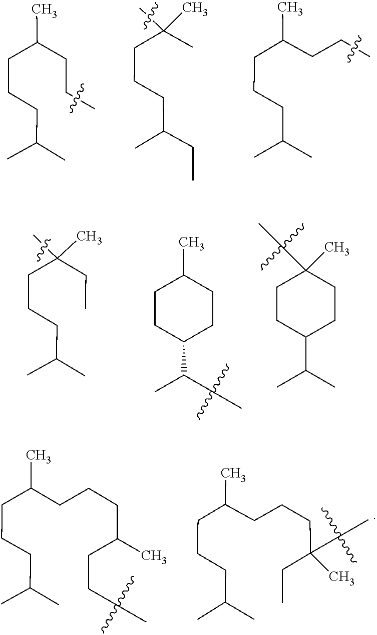

7. The compound of claim 1, wherein R₁ is H.

8. The compound of claim 1, wherein R₁ is —OH.

9. The compound of claim 1, wherein R₂, R₃, and R₄ are each independently selected from H, C₁-C₁₂ alkyl, carboxy (—COOH), carboxylato (—COO⁻), and hydroxy (—OH), wherein said C₁-C₁₂ alkyl is optionally further substituted by one or more groups selected from carboxy (—COOH), carboxylato (—COO⁻), and hydroxy (—OH).

10. The compound of claim 1, wherein group A is an isodecyl group, e.g., selected from 2,4-dimethyloctan-2-yl, 2,6-dimethyl-octan-1-yl, 2,6-dimethyloctan-2-yl, 3,7-dimethyloctan-1-yl, and 3,7-dimethyloctan-3-yl, and optionally wherein R₁ is —H, R₂ is —H, R₃ is —H, and R₄ is —CH₂COOH or —CH₂COO⁻.

11. The compound of claim 1, wherein the compound is selected from the group consisting of: 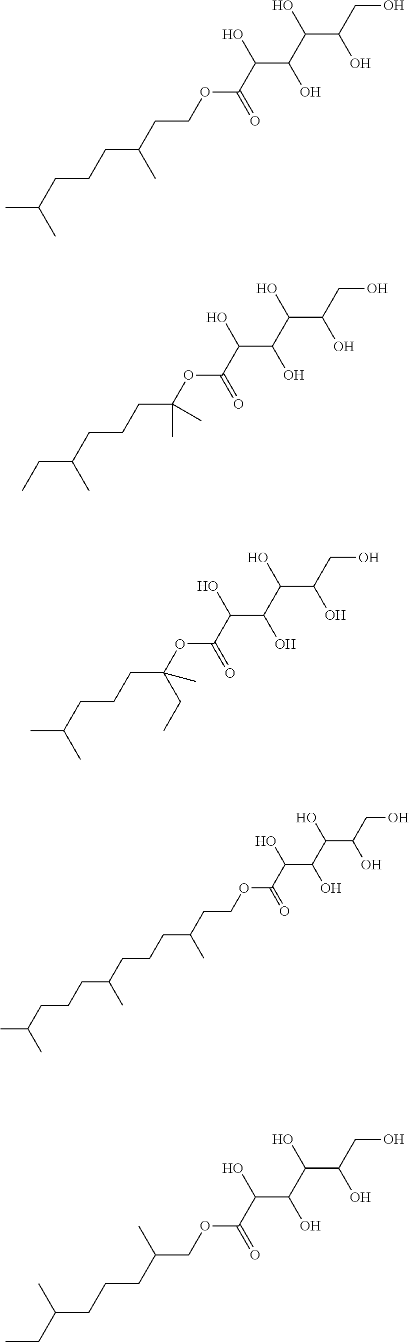 [SMILES: CC(C)CCCC(C)CCOC(=O)C(O)C(O)C(O)C(O)CO ; CCC(C)CCCC(C)(C)OC(=O)C(O)C(O)C(O)C(O)CO ; CCC(C)(CCCC(C)C)OC(=O)C(O)C(O)C(O)C(O)CO ; CC(C)CCCC(C)CCCC(C)CCOC(=O)C(O)C(O)C(O)C(O)CO ; CCC(C)CCCC(C)COC(=O)C(O)C(O)C(O)C(O)CO] 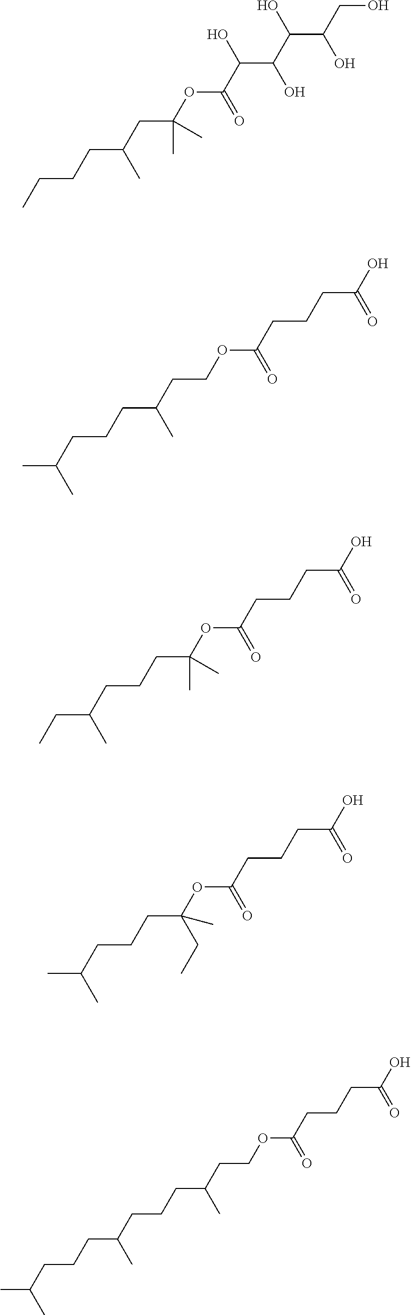 [SMILES: CCCCC(C)CC(C)(C)OC(=O)C(O)C(O)C(O)C(O)CO ; CC(C)CCCC(C)CCOC(=O)CCCC(=O)O ; CCC(C)CCCC(C)(C)OC(=O)CCCC(=O)O ; CCC(C)(CCCC(C)C)OC(=O)CCCC(=O)O ; CC(C)CCCC(C)CCCC(C)CCOC(=O)CCCC(=O)O] 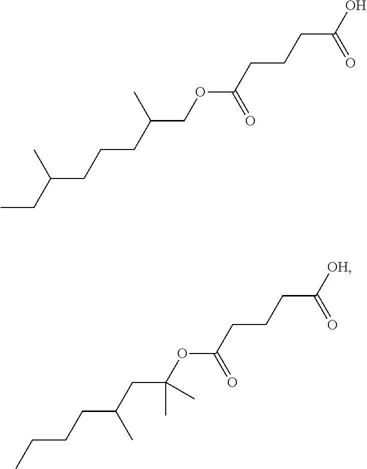 [SMILES: CCC(C)CCCC(C)COC(=O)CCCC(=O)O ; CCCCC(C)CC(C)(C)OC(=O)CCCC(=O)O]
optionally wherein any one or more groups COOH may exist as —CH₂COO⁻; 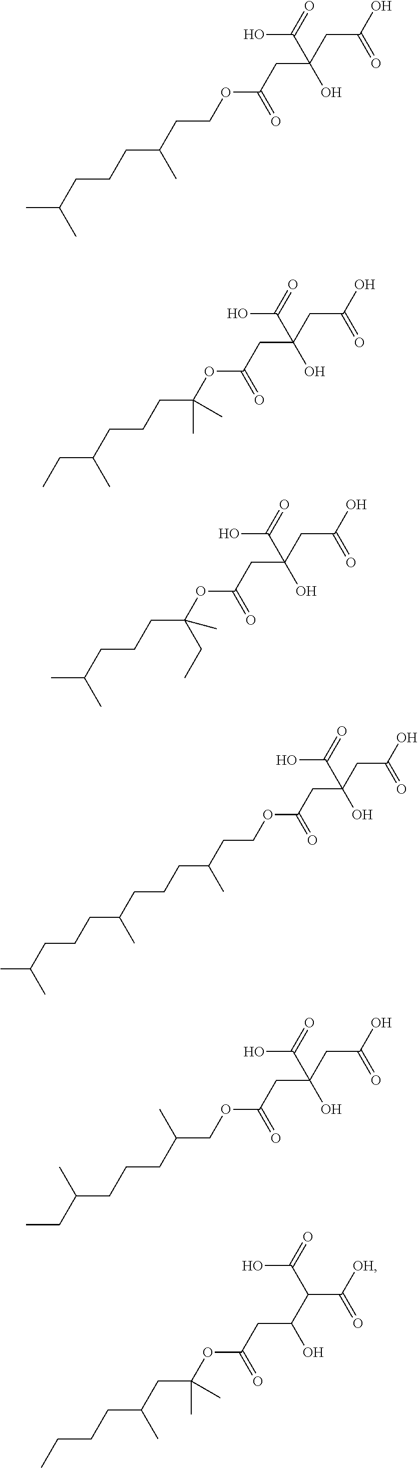 [SMILES: CC(C)CCCC(C)CCOC(=O)CC(O)(CC(=O)O)C(=O)O ; CCC(C)CCCC(C)(C)OC(=O)CC(O)(CC(=O)O)C(=O)O ; CCC(C)(CCCC(C)C)OC(=O)CC(O)(CC(=O)O)C(=O)O ; CC(C)CCCC(C)CCCC(C)CCOC(=O)CC(O)(CC(=O)O)C(=O)O ; CCC(C)CCCC(C)COC(=O)CC(O)(CC(=O)O)C(=O)O ; CCCCC(C)CC(C)(C)OC(=O)CC(O)C(C(=O)O)C(=O)O]
optionally wherein any one or more groups COOH may exist as —CH₂COO⁻; and 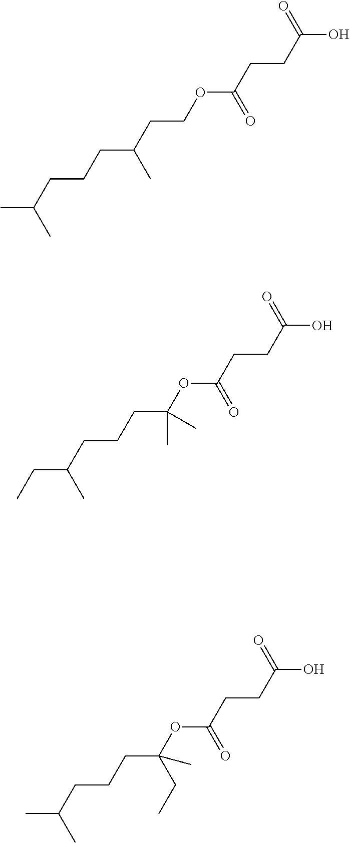 [SMILES: CC(C)CCCC(C)CCOC(=O)CCC(=O)O ; CCC(C)CCCC(C)(C)OC(=O)CCC(=O)O ; CCC(C)(CCCC(C)C)OC(=O)CCC(=O)O] 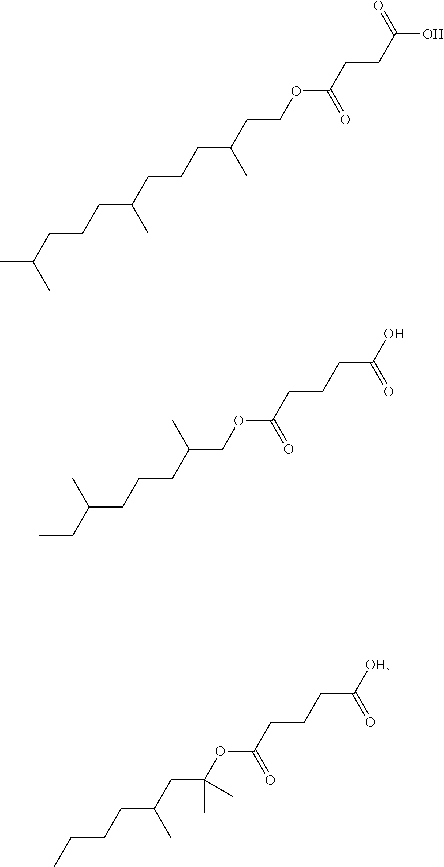 [SMILES: CC(C)CCCC(C)CCCC(C)CCOC(=O)CCC(=O)O ; CCC(C)CCCC(C)COC(=O)CCCC(=O)O ; CCCCC(C)CC(C)(C)OC(=O)CCCC(=O)O]
optionally wherein any one or more groups COOH may exist as —CH₂COO⁻.

12. A method of making the compound of claim 1, wherein the method comprises the step of reacting a compound of the Formula A, with a compound of Formula B, or an ester, activated ester or acyl halide thereof, in a condensation reaction to form the compound of Formula I: 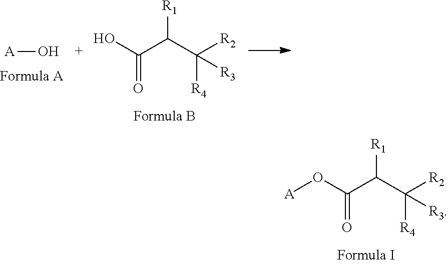
wherein substituents A, R₁, R₂, R₃, and R₄, are as defined in claim 1.

13. The method of claim 12, wherein the reaction comprises a mixture of sulfuric acid and magnesium sulfate, optionally in a hydrocarbon solvent, such as heptane.

14. The method of claim 12, wherein the reaction comprises adding a solid magnesium sulfate/sulfuric acid adduct as catalyst to a mixture of the compound of Formula A and the compound of Formula B, optionally without an additional solvent.

15. A composition comprising a compound according to claim 1, optionally in admixture with one or more pharmaceutically acceptable, cosmetically acceptable, or industrially acceptable excipients or carriers, for example, solvents, oils, surfactants, emollients, diluents, glidants, abrasives, humectants, polymers, plasticizer, catalyst, antioxidant, coloring agent, flavoring agent, fragrance agent, antiperspirant agent, antibacterial agent, antifungal agent, hydrocarbon, stabilizer, or viscosity controlling agent.
